(19) United States
(12) Patent Application Publication
(10) Pub. No.: US 2019/0237162 A1
(43) Pub. Date: Aug. 1, 2019

(54) CONCURRENT SUBTRACTIVE AND SUBTRACTIVE ASSEMBLY FOR COMPARATIVE METAGENOMICS

(71) Applicant: Indiana University Research and Technology Corporation, Indianapolis, IN (US)

(72) Inventor: Yuzhen Ye, Bloomington, IN (US)

(73) Assignee: Indiana University Research and Technology Corporation, Indianapolis, IN (US)

(21) Appl. No.: 16/337,365

(22) PCT Filed: Sep. 30, 2017
(86) PCT No.: PCT/US17/54664

§ 371 Date: Mar. 27, 2019

Related U.S. Application Data
(60) Provisional application No. 62/402,925, filed on Sep. 30, 2016.

(51) Int. Cl. G16B 40/00; G16B 30/00

(52) U.S. Cl. CPC G16B 40/00; G16B 30/00

(57) ABSTRACT

A method for characterizing disease-associated microbial marker genes comprising: counting k-mers of at least two samples; loading k-mers into an array; extracting reads based on differential sequence signatures; and assembling contigs and phylogenetically annotating the contigs.

### CROSS REFERENCE TO RELATED APPLICATION

This application claims priority to U.S. Provisional Application No. 62/402,925, which is entitled “CONCURRENT SUBTRACTIVE AND SUBTRACTIVE ASSEMBLY FOR COMPARATIVE METAGENOMICS,” and was filed on Sep. 30, 2016, the entire disclosure of which is expressly incorporated herein by reference in its entirety.

### GOVERNMENT SUPPORT

This invention was made with government support under AI108888 and 1R01AI108888 awarded by the National Institutes of Health and DBI-0845685 awarded by the National Science Foundation. The government has certain rights in the invention.

### FIELD OF THE DISCLOSURE

This disclosure relates to detection of disease associated genes. More specifically, this disclosure relates to an approach for the characterization of disease-associate microbial marker genes that are conserved across patients.

### BACKGROUND

Metagenomics relies on the direct sequencing of an entire community of microbial organisms, but the results can be hard to disentangle. Microbial communities vary in compositional complexity, from the simplest acid mine drainage microbial community with a few species to more complex microbial communities that may contain hundreds—even thousands—of microbial species (such as the human microbiome). Even though many new methods and tools have been developed for analyzing metagenomics sequences, it remains a great challenge to infer the composition and functional properties of a microbial community from a metagenomic dataset, and to address causal questions, such as the impact of microbes on human health and diseases. Metagenomic assembly (the assembly of metagenomic samples) is one of the challenges. While assembly of a single genome using short reads has improved in recent years, it remains an area of active improvement. In the case of metagenomic datasets, it is difficult for conventional genome assemblers to deal with closely related strains and to distinguish true variations from sequencing errors: using simulated Illumina reads from a 400-genome community, Mende et al. found that relatively few of the reads were assembled, and of the contigs produced, 37% were chimeric. Also, the varied depth of coverage across the individual chromosomes leads to ambiguity in assembly. Finally, the sheer size of metagenomic datasets poses a challenge, as sufficient sequencing must be done to represent ever rarer members of the community. But there is much to be learned by comparing metagenomic datasets sampled from different environments (or hosts): metagenomics can be used to reveal connections between microbes and other aspects of life (such as human health and disease). A recent exemplar is the identification of a connection between microbes and type II diabetes.

Comparative metagenomics studies how environment and/or health correlate with microbial communities phylogenetically and functionally, using either 16S ribosomal RNA data or whole genome shotgun metagenomic sequence data. Early studies compared the genomic diversity and metabolic capabilities across dramatically different metagenomes using barely-assembled sequence data, while recent studies are more concerned with investigating how environmental or health features correlate with metagenomic differences using largely similar metagenomes.

In seeking disease-associated microbes, there are significant compositional variations of microbiota from individual to individual. Regarding this interpatient variability, one strategy is to identify conserved microbial community behaviors in microbiota-associated diseases. Microbial marker gene surveys have been used extensively to reveal the association of microbiota with diseases such as diabetes and Crohn's disease.

Due to the complexity of microbial communities, the de novo partition of metagenomic sequences into specific biological entities remains difficult. To solve this problem, researchers have utilized various features, including compositional features like tetra-nucleotide statistics and coverage signals of genetic sequences. However, the assumptions of those methods are not universally true. For example, methods relying on abundances of genetic sequences are admittedly weak in segregating taxonomically related organisms.

A need therefore exists to accurately identify and detect marker genes for various illnesses and/or diseases.

### SUMMARY

The disclosure provides an algorithm for characterizing disease-associated microbial marker genes that are conserved across patients. The algorithm includes: counting k-mers of groups of samples; loading k-mer counts into a hash table; detecting differential k-mers; loading differential k-mers into an array; extracting reads based on differential k-mers; and assembling contigs and annotating the contigs.

### BRIEF DESCRIPTION OF THE DRAWINGS

The above mentioned and other features and objects of this disclosure, and the manner of attaining them, will become more apparent and the disclosure itself will be better understood by reference to the following description of exemplary embodiments of the disclosure taken in conjunction with the accompanying drawings, wherein:

FIG. 1A shows a flowchart representing a subtractive assembly (SA) algorithm according to the present disclosure;

FIG. 1B shows a flow chart representing a concurrent subtractive assembly (CoSA) algorithm according to an embodiment of the present disclosure;

FIG. 2 is associated with Example 1 and shows a graph representing the fraction of the S. thermophilus LMD-9 genome assembled using subtractive assembly with different k-mer ratio parameters r (2 to 5); an illustrative view of an exemplary game view of the online tele-health platform of FIG. 1;

FIG. 3 is associated with Example 1 and shows a graph representing the percentage of extracted reads from non-differential genomes by subtractive assembly on S1 vs. S2 of Simulation 1;

FIG. 4 is associated with Example 2 and shows a graph representing the comparison of the cumulative contig length of subtractive assembly at different sequencing depths of S. thermophilus LMD-9;

FIGS. 5A and 5B are associated with Examples 3 and 4 and show graphs representing the comparison of the cumulative contig length between subtractive assembly and direct metagenomics assembly of R. palustris HaA2 assembled by IDBA-UD and Meta Velvet respectively;

FIG. 6 is associated with Example 3 and shows an example of the reduced fragmentation of contigs given by subtractive assembly;

FIG. 7 is associated with Example 4 and shows a comparison of the cumulative contig length between subtractive assembly and direct assembly;

FIG. 8 is associated with Example 6 and shows compositional differences in Streptococcus species between the Type 2 Diabetes (T2D) and Normal Glucose Tolerant (NGT) group;

FIGS. 9A and 9B are associated with Example 7 and show the truB-ribF operon identified by subtractive assembly as associated with T2D;

FIG. 10 is associated with Example 8 and shows upper and lower subfigures that refer to read extraction for one of the samples of population 1 and 2, respectively. The x-axis shows the 5 different species; fac: Ferroplasma acidarmanus fer1, lga: Lactobacillus gasseri ATCC 33323, ppe: Pediococcus pentosaceus ATCC 25745, pmn: Prochlorococcus marinus NATL2A, ste: Streptococcus thermophilus LMD-9. Bars of different colors indicate separate runs of CoSA using different parameters or different number of samples while the grey bars (rightmost bar on each specie) indicate simulated reads for each genome. The y-axis shows the number of reads;

FIG. 11 is associated with Example 11 and shows Neighbor-Joining topology of a gut microbiome in liver cirrhosis that was used in discovery phase;

FIG. 12 is associated with Example 10 and shows the accuracy of prediction based on selected marker genes using 10-fold cross validation;

FIG. 13 is associated with Example 7 and shows the abundance difference of the genes encoding beta-galactosidase between T2D and normal microbiomes (NGT), where the abundance was measured as the number of reads that can be mapped to significantly T2D-enriched beta-galctosidase-encoding genes per billion reads; and

FIG. 14 is a graph associated with Example 8 and shows an evaluation of the assembly quality of differential genomes where CoSA-10 means that 10 samples were used in testing CoSA and CoSA-5 means that 5 samples were used in testing CoSA.

Corresponding reference characters indicate corresponding parts throughout the several views. Although the drawings represent embodiments of the present disclosure, the drawings are not necessarily to scale and certain features may be exaggerated in order to better illustrate and explain the present disclosure. The exemplification set out herein illustrates exemplary embodiments of the disclosure, in various forms, and such exemplifications are not to be construed as limiting the scope of the disclosure in any manner.

### DETAILED DESCRIPTION

The embodiment disclosed below is not intended to be exhaustive or limit the disclosure to the precise form disclosed in the following detailed description. Rather, the embodiments are chosen and described so that others skilled in the art may utilize its teachings.

One of ordinary skill in the art will realize that the embodiments provided can be implemented in hardware, software, firmware, and/or a combination thereof. Programming code according to the embodiments can be implemented in any viable programming language such as C, C++, HTML, XTML, JAVA or any other viable high-level programming language, or a combination of a high-level programming language and a lower level programming language.

As used herein, the modifier “about” used in connection with a quantity is inclusive of the stated value and has the meaning dictated by the context (for example, it includes at least the degree of error associated with the measurement of the particular quantity). When used in the context of a range, the modifier “about” should also be considered as disclosing the range defined by the absolute values of the two endpoints. For example, the range “from about 2 to about 4” also discloses the range “from 2 to 4.”

Subtractive Assembly (SA)

A subtractive assembly is a de novo method to compare metagenomes through metagenomics assemblies, aiming to achieve better assembly of differential genomes for downstream analysis (e.g., to infer potential microbial markers associated with a disease). For two or more metagenomes, reads that constitute the compositional difference are extracted from each metagenome based on sequence signatures. For example, k-mers that occur 10 times more frequently in one dataset than in the other are “signatures” that constitute the genomic difference; reads containing these signatures are likely to be from genomes that are more abundant or even unique in one of the two metagenomes.

After read filtering, the complexity of the metagenome data sets can be greatly reduced, such that metagenome assembly using the extracted distinctive reads can be improved due to reduction in both biological diversity and data size. The compositional and functional difference of metagenomes can thus be characterized by the better-assembled contigs obtained from subtractive assembly.

For k-mer-based methods, one step is the counting and storing of all k-mers. In one exemplary embodiment, a k-mer counting algorithm includes BFCounter, version 0.2 created by Arash Partow and distributed under the Common Public License, which adopts a Bloom filter making BFCounter memory efficient and suitable for comparative metagenomics. The Bloom filter is a probabilistic data structure for determining whether an element belongs to a sparse set, using a number of hash functions to map the elements to the fixed bit space of the filter.

The C++ code of BFCounter is modified to output reads with distinctive signatures. Functions are added to BFCounter for counting k-mers for multiple samples, comparing k-mer counts between two groups of samples to generate signature k-mers (i.e., differential k-mers), and extracting sequencing reads that contain signature k-mers. With simulated metagenomic datasets, it is shown that subtractive assembly can both effectively extract the reads from genomes that cause the compositional differences between metagenomes, and improve metagenomic assembly for these genomes.

Subtractive assembly reduces the complexity of metagenome assembly by filtering out reads that can be classified to known genomes, assuming that they are not of interest. The subtractive assembly approach takes advantage of the availability of metagenomic datasets of the same community under different conditions. When the differences between two (groups of) metagenomes are of interest, the differences in the metagenomes can be assembled by filtering out reads that are likely to have been sampled from species that are common to both samples. In other words, the method is independent of reference genomes.

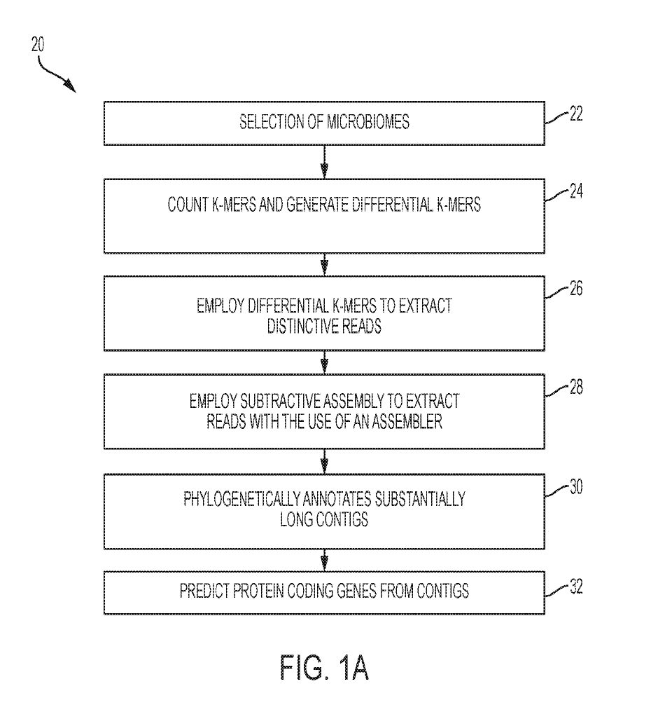

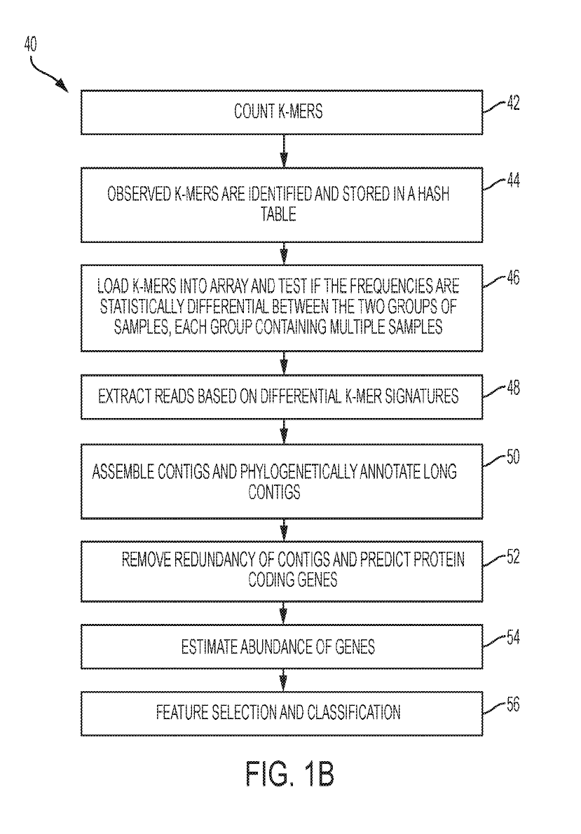

In operation, method 20 operates as shown in FIG. 1A. As shown in block 22, at least two different microbiomes are selected. In one exemplary embodiment, one microbiome is selected from a diseased patient, and the other microbiome is selected from a healthy patient. At block 24, k-mers are counted. In an exemplary embodiment, a k-mer counting algorithm may be used. In a further exemplary embodiment, the k-mer counting algorithm includes BFCounter which adopts a Bloom filter. As mentioned earlier, the C++ code of BFCounter is modified based on the principle of the BFCounter to rule out singletons of all k-mers encountered in order to output reads with distinctive signatures.

A Bloom filter B and a simple hash table T are adapted to store and count k-mers. The Bloom filter B is used to store all existing k-mers, of which k-mers observed twice or better are inserted into the hash table T. With the information stored in hash table T, the distinctive sequence signatures can be calculated for each metagenome. To detect compositional differences of metagenomes, a k-mer ratio parameter is employed to filter for k-mers that are more abundant or unique in a metagenome. For example, if the k-mer ratio parameter is set to 10, then k-mers that occur at least 10 times more frequently in metagenome A than metagenome B will be retained as differential k-mers for metagenome A.

At block 26, differential k-mers generated from block 24 are used to identify distinctive reads based on a set requirement. In one exemplary embodiment, distinctive reads are reads containing at least a certain percentage (e.g., default 50%) of differential k-mers. Reads that meet the set requirement are extracted and employed for metagenomics assembly.

At block 28, a subtractive assembly algorithm is used to extract the aforementioned reads with the use of an assembler. In one embodiment, the assembler, IDBA-UD, version 1.0.9 as described in Yu Peng, Henry C. M. Leung, S. M. Yiu, and Francis Y. L. Chin; IDBA-UD: a de novo assembler for single-cell and metagenomic sequencing data with highly uneven depth; Bioinformatics (2012) 28 (11): 1420-1428 first published online Apr. 11, 2012, doi:10.1093/bioinformatics/bts174 may be adopted as the metagenomics assembler. In a further exemplary embodiment, the default settings for IDBA-UD were used (e.g., minimum k-mer size of 20 and a maximum k-mer size of 100, with 20 increments in each iteration). In another exemplary embodiment, MetaVelvet, version 1.1.01 as described in Namiki T., Hachiya T., Tanaka H., Sakakibara Y. (2012). MetaVelvet: an extension of Velvet assembler to de novo metagenome assembly from short sequence reads. Nucleic Acids Res. 40:e155 10.1093/nar/gks678, and MEGAHIT, version 0.2.1 as described in “MEGAHIT: an ultra-fast single-node solution for large and complex metagenomics assembly via succinct de Bruijn graph.” Dinghua Li, Chi-Man Liu, Ruibang Luo, Kunihiko Sadakane, Tak-Wah Lam. Bioinformatics. 2015 May 15; 31(10): 1674-1676. Published online 2015 Jan. 20. doi: 10.1093/bioinformatics/btv033, are other assemblers that may be used.

When the subtractive assembly algorithm is applied to metagenomic samples, a small k-mer ratio cutoff (e.g., 2) may be selected, due to the unknown degree of compositional difference between the groups of samples being compared. Alternatively, differential reads can be iteratively extracted using a series of k-mer ratio cutoffs. For example, for gut metagenomic datasets that were studied, a maximum ratio was set to 10 and a minimum ratio was set to 2, with a step value of 2. For example, k-mers that are more frequent in one group than another are identified, and the distinctive reads were extracted by iteratively varying the k-mer ratio, starting from a k-mer ratio of 10 and proceeding stepwise until reaching a k-mer ratio of 2. The stratification by iterative assembly provides more information of the compositional difference between two metagenomes, without any prior knowledge.

In addition to the iterative extraction, k-mers that occur in one of the groups of samples are separately extracted, i.e., “unique k-mers.” The unique k-mers are first identified in each group and the corresponding distinctive reads are extracted.

At block 30, contigs are assembled from the differential reads, and contigs that are substantially long are phylogenetically annotated by query against bacteria genomes. In one exemplary embodiment, contigs that are at least 300 nucleotides long were phylogenetically annotated by query against the bacterial genomes (both complete and draft genomes) deposited in the National Center for Biotechnology Information (NCBI) through BLAST searches. BLAST results were then used for the assignment of lowest common ancestor (LCA) by MEGAN, version 4, as described in Huson, Daniel H. et al. “MEGAN Analysis of Metagenomic Data.” Genome Research 17.3 (2007): 377-386. PMC. Web. 28 Sep. 2016, with a minimum bit score of 80 and minimum contig support of 5.

Protein coding genes are then predicted from the contigs using a program (e.g., an application for finding (fragmented genes) in short reads) as indicated at block 32. In one exemplary embodiment, the program used to predict the protein coding genes from the contigs is FragGeneScan, available from Indiana University. An exemplary program includes FragGeneScan, version 1.16. The protein coding genes that are predicted are genes that remain from subtractive assembly, and a gene is considered belonging to this category if there is no equivalent gene that covers at least 20% of the gene with 90% or higher sequence identity based on a protein similarity search in the direct assemblies of any individual metagenome. In an exemplary embodiment, the protein similarity search is conducted by an application for searching short DNA sequences (reads) or protein sequences against protein database. In an exemplary embodiment, RAPSearch2 available at Indiana University is used. In a further exemplary embodiment, RAPSearch2, version 2.20 is used. The genes are then assigned to functional categories, including for example, SEED subsystems. In one exemplary embodiment, myRAST, version 36 is used for the SEED subsystem annotation.

To further validate the differential genes, the original short reads of each sample are mapped onto the genes that are enriched in a diseased patient and normalize the coverage by the total number of reads in each sample. Based on the coverage of each differential gene in each sample, the significance of each candidate differential gene is tested by computing a one-sided p-value, using the Wilcoxon Rank Sum test function and correcting for multiple testing using a false discovery rate (q-value) computed by the tail area-based method of the R ‘fdrtool’ package, version 1.2.15, as described in Strimmer, K. 2008. A unified approach to false discovery rate estimation. BMC Bioinformatics 9: 303; and Strimmer, K. 2008. fdrtool: a versatile R package for estimating local and tail area-based false discovery rates. Bioinformatics 24: 1461-1462.

As discussed further below, both simulated and real metagenomes show that subtractive assembly improves the assembly of the differential genome between two metagenomes in comparison, and facilitates downstream analysis. If the short reads from many genomes are directly assembled and annotated, it takes a tremendous amount of computational resources, as well as degrading the quality of the assembly. As a result, traditional comparative metagenomic approaches assemble each of the metagenomic samples independently, and then compare groups of samples by the common features shared among samples in each group.

By contrast, subtractive assembly focuses on the compositional difference of the metagenome sets to be compared and therefore is well suited for largescale comparative studies. Subtractive assembly is able to consider a large number of samples simultaneously, which can also improve the assembly of differential genes, providing a complementary solution to existing comparative metagenomic approaches.

The iterative subtractive assembly strategy deals with the typical situations where the compositional differences between metagenomes are unknown. An advantage of iterative subtractive assembly is that it samples a spectrum of differences, aiding the assembly of genomes that are differential at various levels. If a user is interested in a certain degree of difference, a fixed k-mer ratio cutoff can be used in the subtractive assembly. In sum, the analysis of T2D-hosted metagenomes discussed further below indicates that subtractive assembly has a greater ability to detect differences, than did previous analysis of the same data sets via direct assembly.

Moreover, subtractive assembly utilizing the reads that represent the compositional difference substantially reduce the complexity of the datasets and greatly improve the quality of the resulting assemblies, facilitating identification of compositional and functional differences between microbiomes. Application of SA to the T2D datasets results in a large collection of genes that are uniquely found in the T2D-associated gut microbiomes, but which had not previously been identified.

Concurrent Subtractive Assembly (CoSA)

Concurrent Subtractive Assembly (CoSA) is an algorithm designed to identify short reads that make up conserved/consistent compositional differences across multiple samples based on sequence signatures (k-mer frequencies) and then assembles the differential reads, aiming to reveal the consistent differences between two groups of metagenomic samples (e.g., metagenomes from cancer patients vs. metagenomes from healthy controls).

Instead of using fold change of k-mers, CoSA detects differential genomes by testing k-mer frequencies with a Wilcoxon rank-sum test as discussed further herein. CoSA also employs k-mer frequencies concurrently from multiple samples for each group in comparison.

CoSA can be implemented in various programming codes. In one exemplary embodiment, CoSA is implemented in C++. Because CoSA employs k-mer frequencies from individual samples, it introduces a new dimension for different samples and therefore increases the requirement of computational resources, especially for large cohort of datasets such as the T2D datasets. To reduce the running time and memory usage, CoSA is implemented with multiple threading. Also, counts of k-mers are written to disk and then loaded back in batches for the detection of differential k-mers (since it is difficult to load all k-mer counts into the memory at the same time).

FIG. 1B shows a flowchart for the analysis, which is based on CoSA, for characterization of disease related sub-metagenomes. As shown, sequencing data undergoes CoSA algorithm, assembly, and prediction steps as discussed in further detail below.

Referring now to FIG. 1B, a CoSA algorithm 40 is shown. At block 42, k-mers are counted. Generally, the sheer size of the datasets prove to be a fundamental challenge as it tends to be time consuming to count billions of k-mers in metagenomic samples. As such, a program is used to count k-mers. In one embodiment, KMC-2, as described in KMC 2: fast and resource-frugal k-mer counting; Sebastian Deorowicz, Marek Kokot, Szymon Grabowski, Agnieszka Debudaj-Grabysz; Bioinformatics. 2015 May 15; 31(10): 1569-1576. Published online 2015 Jan. 20. doi: 10.1093/bioinformatics/btv022, is used to count k-mers in which a maximal value of a counter (the cs flag) is set. A higher counter value helps identify the more frequently observed differential k-mers by using a larger cut-off value. Each counter can be stored using a 16-bit unsigned integer, which demands reasonable amount of memory or disk space when we are dealing with billons of k-mers. Meanwhile, k-mers occurring less than 2 times are excluded based on the fact that large number of singletons are products from sequencing errors. In an exemplary embodiment, the counter (cs flag) is set to 65,536.

After k-mer counting is completed at block 42, the CoSA algorithm proceeds to block 44 where observed k-mers (outputs) of the counter program (e.g., KMC 2) are identified and stored in a hash table. In one embodiment, a library (e.g., Lilbcuckoo, as described in Li et al, 2014. Algorithmic improvements for fast concurrent Cuckoo hashing in Proceedings of the Ninth European Conference on Computer Systems Article No. 27) is used to provide a high-performance concurrent hash table, by which the hash table can be efficiently updated using multiple threads. With the k-mers in the hash table, the CoSA algorithm accesses the outputs of the counter program (e.g., KMC-2) and records the counters of the k-mers onto disk based on the k-mers orders in the hash table for every sample. By storing the counters on the disk, the counting information of k-mers can be loaded in batches, which significantly reduces the maximal memory requirement for recording the counters for all k-mers in every sample.

Proceeding to block 46, the CoSA algorithm, by default, loads a number of k-mers (e.g., le7 or 10⁷ or 1×10⁷ k-mers) into a two-dimensional array each time and tests if the frequencies of each k-mer are differential between the two groups of samples. Due to the large number of k-mers and memory constraints of the program, testing is done iteratively until all the k-mers are processed. To compare k-mers in different metagenomic samples, the frequency of each k-mer in each metagenomic sample is calculated. In case the frequency of a k-mer is substantially small, the frequency of each k-mer is computed in the number of occurrences per million k-mers. The normalized frequencies are then statistically analyzed to determine which k-mers are to be classified as differential k-mers. The statistical analysis is used to detect k-mers that have different frequencies in one group of the samples (e.g., the patient group) than the other group of samples (e.g., the healthy control) with statistical significance. The k-mers that pass the test (e.g., p-value is set to 0.05) are identified as differential k-mers. In one embodiment, a Wilcoxon Rank Sum (a nonparametric test) test is used, in which the mannwhitneyutest function from a numerical analysis and data processing library is employed with a p-value cutoff of 0.05. In a further embodiment, the numerical analysis and data processing library includes ALGLIB library, version 3.8.2. It is within the scope of the present disclosure that alternate versions of the AGLIB library may be used.

Reads that are composed of differential k-mers tend to be from differential genomes. Thus, once the differential k-mers are identified, the CoSA algorithm proceeds to block 48, in which reads are extracted from the sequencing data based on differential k-mers. Differential reads in each sample are extracted by differential k-mers by using a voting strategy, i.e., a voting threshold. For example, a voting threshold of 0.5 means that a read is considered to be differential if 50% of its k-mers belong to differential k-mers. Users may change this parameter according to their own applications of CoSA. In an exemplary embodiment, the voting threshold is between 0.3 and 0.8.

Some k-mers are extremely abundant in the extracted reads file—these k-mers may be from reads sampled from abundant species that are common across many samples. When the differential reads contain these k-mers, the distribution of k-mers can be skewed. The reads redundancy can be reduced by excluding reads that contain highly abundant k-mers at block 49. The reads redundancy removal relies on a list of highly abundant k-mers prepared based on k-mer counts. A read is determined to be redundant if it contains many k-mers on the abundant k-mer list. That is, for each read, the fraction of abundant k-mer (over all k-mers) is computed and if the fraction is smaller than a random number between 0 and 1 generated by the program, the read is retained. Otherwise, the read is discarded. In this way, a read that has a higher ratio of abundant k-mers will have a higher chance to be discarded.

Following read extraction, the CoSA algorithm 40 moves to block 50 where a metagenomic assembler can be utilized similar to subtractive assembly described earlier to assemble contigs. In one exemplary embodiment, the assembler, IDBA-UD, version 1.0.9, as described in Yu Peng, Henry C. M. Leung, S. M. Yiu, and Francis Y. L. Chin; IDBA-UD: a de novo assembler for single-cell and metagenomic sequencing data with highly uneven depth; Bioinformatics (2012) 28 (11): 1420-1428 first published online Apr. 11, 2012, doi:10.1093/bioinformatics/bts174, may be used since IDBA-UD returns longer contigs with higher accuracy by taking into consideration uneven sequencing depth of metagenomic sequencing technologies. In a further embodiment, the default options for IDBA-UD's parameter settings were used: a minimum k-mer size of 20 and maximum k-mer size of 100, with 20 increments in each iteration. In an alternate embodiment, co-assembly of the extracted reads (extracted reads from the individual samples were pooled) using MEGAHIT, version 0.2.1, as described in “MEGAHIT: an ultra-fast single-node solution for large and complex metagenomics assembly via succinct de Bruijn graph.” Dinghua Li, Chi-Man Liu, Ruibang Luo, Kunihiko Sadakane, Tak-Wah Lam. Bioinformatics. 2015 May 15; 31(10): 1674-1676. Published online 2015 Jan. 20. doi: 10.1093/bioinformatics/btv033, may be used depending on memory availability and program speed as MEGAHIT uses less memory and is significantly faster than IDBA-UD, for large cohorts of sample. MEGAHIT tended to give slightly fewer genes as compared to IDBA-UD for the same dataset. It is within the scope of the present disclosure that alternate assemblers (e.g., metaSPAdes) may be used.

After assembly, long contigs (e.g., at least 300 base pairs long) are phylogenetically annotated by querying against the bacterial genomes deposited in the National Center for Biotechnology Information (NCBI). In one embodiment, a local alignment search tool for finding regions of similarity between biological sequences, comparing nucleotide or protein sequences to sequence databases, and calculating the statistical significance is used. for the assignment of lowest common ancestor (LCA). In a further embodiment, BLAST searches, available at the NCBI are used, and the BLAST results are used for the assignment of lowest common ancestor (LCA) by MEGAN, version 4, as described in Huson, Daniel H. et al. “MEGAN Analysis of Metagenomic Data.” Genome Research 17.3 (2007): 377-386. PMC. Web. 28 Sep. 2016, with a minimum score of 80 and minimum contig support of 5.

After annotation, at block 50, the extracted reads for each sample are assembled separately by IDBA-UD, version 1.0.9, as described in Yu Peng, Henry C. M. Leung, S. M. Yiu, and Francis Y. L. Chin; IDBA-UD: a de novo assembler for single-cell and metagenomic sequencing data with highly uneven depth; Bioinformatics (2012) 28 (11): 1420-1428 first published online Apr. 11, 2012, doi:10.1093/bioinformatics/bts174. After assembly, the contigs for different samples in each group are pooled together and the redundancy is removed as shown in block 52. In one exemplary embodiment, a genome assembler (e.g., Minimus—included in AMOS version 3.1.0 and created by Sommer et al), is used to pool together and remove the redundancies of the contigs.

Also at block 52, protein coding genes are predicted from the contigs using a program. In one exemplary embodiment, the program uses to predict the protein coding genes from the contigs is FragGeneScan (e.g., version 1.30).

Once redundancies are removed and protein coding genes are predicted, the abundance of the genes are estimated as shown at block 54. To estimate the abundance of the genes, all the reads from each sample are aligned against the gene set, as assembled by CoSA and annotated by an application for finding (fragmented) genes in short reads (e.g., FragGeneScan available at Indiana University—FragGeneScan, version 1.16), by using a reads mapping tool. In an exemplary embodiment, Bowtie 2 (version 2.2.5) is used to map reads onto the genes and then the reads counts are used to compute a gene's abundance. In a further embodiment, for simplicity, a gene's abundance is counted by uniquely and multiplely mapped reads. The contribution of multiplely mapped reads to a gene was computed according to the proportion of the multiplely mapped read counts divided by the gene's unique abundance. The read counts are then normalized per kilobase of gene per million of reads in each sample.

In a further exemplary embodiment, further filtering for differential genes can be performed by running a Wilcoxon Rank Sum Test for the gene profile matrix between the patient and the healthy control groups with a proper Benjamini-Hochberg test correction (e.g., a q-value less than le-5).

With the gene profile matrix of the gene biomarkers, a classifier can be created to discriminate diseased patients from healthy controls as shown at block 56. In one exemplary embodiment, a Recursive Feature Elimination (RFE) method in a pathClass R package, version 0.94, as described in Johannes M, et al. (2010). Integration Of Pathway Knowledge Into A Reweighted Recursive Feature Elimination Approach For Risk Stratification Of Cancer Patients. Bioinformatics, for feature selection may be used. However, it is contemplated that in alternate embodiments, other suitable programs may be used. Since the RFE algorithm is based on support vector machines (SVM), in some exemplary embodiments, a SVM classifier can be trained during the process of recursive feature selection by RFE.

In an alternate embodiment, an L1-based feature selection method in the “scikit learn” python package is used to select genes. After the feature selection, Random Forest (RF, e.g., 10 trees) and Support Vector Machine (SVM, e.g., linear kernels) are used to build classifiers. RF has been shown to be a suitable model for exploiting non-normal and dependent data such as metagenomic data, and it has been used for prediction of T2D (Type 2 Diabetes). SVMs have been used in computational biology due to their high accuracy and ability to deal with high-dimensional and large datasets. As discussed further herein, the predictive power of a model is evaluated as the Area Under Curve (AUC) using a tenfold cross-validation method.

### EXAMPLES

Type 2 diabetes is one of the many diseases that have an associated microbial “profile”: type 2 diabetes (T2D) is associated with increased levels of streptococci, lactobacilli and Streptococcus mutans in oral samples; Lactobacillus in gut microbiota is linked to obesity in humans, and weight gain for newborn ducks and chick. It has also been found that Karlsson et al. four Lactobacillus species and Streptococcus mutans are enriched in the gut microbiota of European women with T2D, using a large cohort of gut microbiome datasets. The subtractive assembly method (SA) was applied to these gut metagenomes to see if the previous results could be replicated, and perhaps furthered. SA revealed new phylogenetic and functional features of the gut microbial communities associated with T2D.

Examples 1-8 are directed to SA datasets and findings, and Examples 9-12 are directed to CoSA datasets and findings.

For Examples 1-8, two simulations were carried out to test if the SA approach can efficiently detect compositional differences between metagenomes (and the minimum abundance ratio for the difference to be detected), and improve assembly quality, especially when closely-related species co-exist in a community. Datasets for human patients were analyzed as well.

In Simulation 1, three groups of metagenomic datasets were simulated using five bacterial genomes from the FAMeS dataset: Ferroplasma acidarmanus fer1, Lactobacillus gasseri ATCC 33323, Pediococcus pentosaceus ATCC 25745, Prochlorococcus marinus NATL2A, and Streptococcus thermophilus LMD-9. MetaSim (version 0.9.1, as described in Richter et al. (2008): MetaSim—A Sequencing Simulator for Genomics and Metagenomics. PLoS ONE 3(10): e3373. doi:10.1371/journal.pone.0003373) was used to simulate reads from the genomes. In each group, the first sample (51) was compared to each of the remaining samples in the same group for SA. The relative abundances of the five genomes in each sample are shown in Table 1 further below. In these samples, the abundances of the Streptococcus thermophilus genome and another genome were changed while keeping the ratio of relative abundance for the S. thermophilus genome in the range of 2 to 16. This enables proper evaluation of SA to determine whether SA can effectively detect the compositional difference between metagenomes by focusing on a single genome (S. thermophilus). Iterative subtractive assembly strategy was applied to analyze this set of simulated datasets (k-mer ratio parameter r was set to be 2, 3, 4 and 5). After the SA, the fraction of the S. thermophilus genome covered by contigs are calculated using QUAST, version 2.3, as described in Gurevich A, Saveliev V, Vyahhi N, Tesler G. 2013. QUAST: quality assessment tool for genome assemblies. Bioinformatics Oxf Engl 29:1072-1075. doi:.10.1093/bioinformatics/btt086, and MUMmer, version 3.0, as described in Delcher, Arthur L. et al. “Fast Algorithms for Large-Scale Genome Alignment and Comparison.” Nucleic Acids Research 30.11 (2002): 2478-2483. Print. In all the samples, the sequencing depth of the Streptococcus genome was designed to be between 30× and 40×.

In Simulation 2, a pair of metagenomic samples (S1 and S2) was simulated using five different Rhodopseudomonas palustris strains as shown in Table 2 discussed further below. The R. palustris HaA2 genome is dominant in sample 1 (51) and is the focus of this simulation. The k-mer ratio parameter was set to r=2 for SA (51 subtracted by S2). Sample 1 was also used for direct metagenomic assembly using IDBA-UD. Assemblies from both SA and direct assembly were compared to the reference genomes using QUAST. Various metrics were used for assembly evaluation, including the cumulative length of contigs, N50, and size of the largest contig.

After the simulations, SA was used on datasets from humans. The large collection of gut metagenomic datasets derived from two groups of 70-year-old European women, one group of 50 with type 2 diabetes (T2D) and the other a matched group of healthy controls (NGT group; 43 participants) was selected. This collection of metagenomes is ideal for testing SA approach because only two groups were involved (T2D vs. healthy), and each group contains many large metagenomics datasets. The T2D samples and NGT samples were pooled, separately, for SA.

### Example 1

K-mer-counting-based extraction of differential reads was tested using simulated metagenomic samples composed of five bacterial genomes in three groups of 5, 4, and 3 samples as shown in Table 1 below.

TABLE 1

| | Group 1 | | | | | Group 2 | | | | Group 3 | | |
|---|---|---|---|---|---|---|---|---|---|---|---|---|
| | S1 | S2 | S3 | S4 | S5 | S1 | S2 | S3 | S4 | S1 | S2 | S3 |
| Ferroplasma acidarmanus fer1 | 1¹ | 16 | 1 | 1 | 1 | 1 | 12 | 1 | 1 | 1 | 10 | 1 |
| Lactobacillus gasseri ATCC 33323 | 2 | 2 | 16 | 2 | 2 | 2 | 2 | 12 | 2 | 2 | 2 | 10 |
| Pediococcus pentosaceus ATCC 25745 | 4 | 4 | 4 | 16 | 4 | 4 | 4 | 4 | 12 | 4 | 4 | 4 |
| Prochlorococcus marinus NATL2A | 8 | 8 | 8 | 8 | 16 | 8 | 8 | 8 | 8 | 8 | 8 | 8 |
| Streptococcus thermophilus LMD-9 | 16 | 1 | 2 | 4 | 8 | 12 | 1 | 2 | 4 | 10 | 1 | 2 |
| RA ratio (S₁/S [symbol] ,/1 = 1)² | | 16³ | 8 | 4 | 2 | | 12 | 6 | 3 | | 10 | 5 |

¹Relative Abundance (RA) of the F. acidarmanus species in sample S1.
²Relative abundance of the S. thermophilus genome in S1 relative to S2, S3 and so on. Pairs of datasets (S1 in each group, and another one in the same group) were subject to subtractive assembly.
³The relative abundance of the S. thermophilus genome in S1 versus S2. [symbol]
indicates data missing or illegible when filed

In each group, S1 has a uniquely large proportion of S. thermophilus reads. For each of the groups, sample 1 (S1) was subtracted by each of the other samples (S2, S3, etc.) and the remaining reads were used for assembly. As can be seen, the fold change of the S. thermophilus genome ranges from 2 to 16 as shown in Table 1 above.

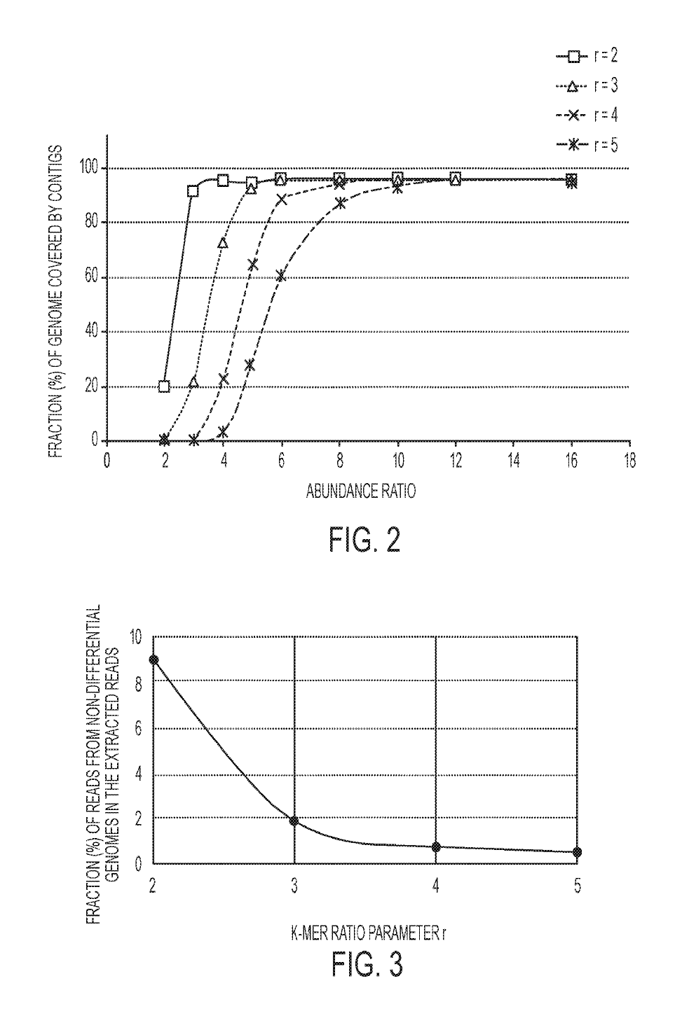

The effects of assembly coverage of S. thermophilus reference genome changes when the parameters, including the actual abundance ratio of the genome in two metagenomes (or the fold change) and the k-mer ratio threshold used in the subtractive assembly, are changed was analyzed as shown in FIG. 2. The results, as shown in FIG. 2, suggest that subtractive assembly can effectively detect the differential genome when the abundance ratio of the genome between two samples is about two times (or greater) of the k-mer ratio threshold (parameter r). However, when r decreases to a value less than 2, significantly more reads from non-differential genomes are also extracted and subtractive assembly loses its advantageous purpose (see FIG. 3). For instance, 97.84% (581,047 out of 593,858) of the reads from S. thermophilus LMD-9 were extracted and 95.03% of the genome is covered by contigs when r=2 and the simulated abundance of the Streptococcus genome is four times different in abundance between the two datasets. Based on the data shown, a k-mer ratio threshold needs to be set to r=R/2 to effectively assemble a genome that is about R times more abundant in sample A than B, using subtractive assembly (i.e., sample A subtracted by sample B). The simulation also suggests that the subtractive assembly approach can effectively capture genes with abundance changes of 3 fold or more.

### Example 2

As shown above, SA can effectively recover reads originating from differential species between metagenomes. However, due to the random nature of shotgun sequencing, some regions of differential species may lack reads and are therefore poorly assembled, especially when the sequencing depth is not high. In this Example, simulated datasets were used with varying sequencing depths to demonstrate the impact of sequencing depth on the performance of SA, using the same population structure as S1 or S4 from Group 1 in Simulation 1 of Table 1. Five pairs of datasets were synthesized in which the sequencing depth ranges from 1× to 20× as shown in Table 2 below.

TABLE 2

| Sequencing Depth | | Base | Extracted | Assembled |
|---|---|---|---|---|
| S1¹ | S4 | Coverage² (%) | Reads³ (%) | Genome⁴ (%) |
| 4x | 1x | 82.15 | 86.69 | 31.72 |
| 8x | 2x | 86.96 | 88.93 | 67.31 |
| 12x | 3x | 90.58 | 93.15 | 83.72 |
| 16x | 4x | 92.96 | 95.53 | 90 92 |
| 20x | 5x | 94.79 | 96.91 | 93.82 |

¹The community structures of S1 and S4 are the same as in Simulation 1 (Grooup 1 in Table 1).
²Expected percentage of bases with >=2 times sequencing coverage in S1 than in S4.
³Percentage of reads extracted from the simulated S. thermophilus genome in S1.
⁴Fraction of the genome assembled using the extracted reads by our subtractive assembly approach.

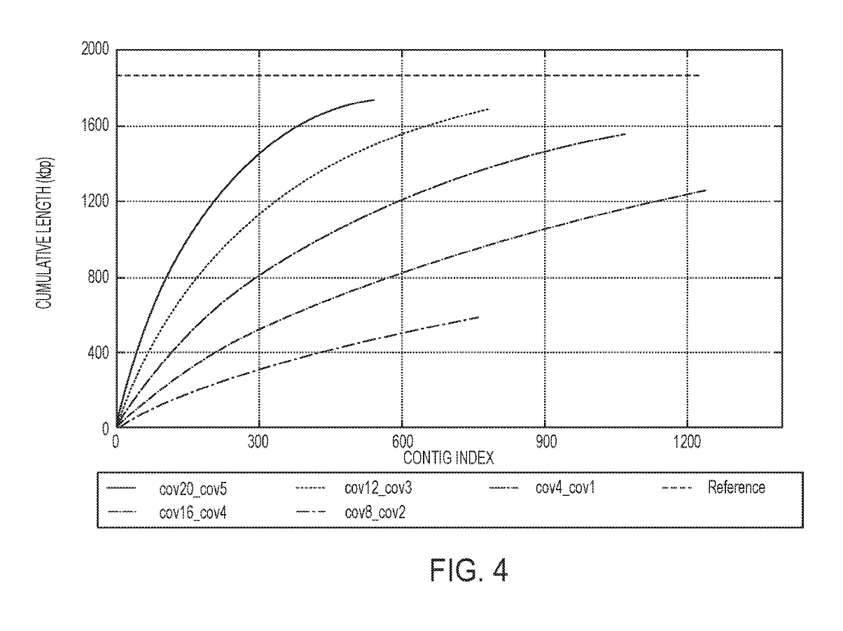

The sequencing depth for S1 ranges from 4 times to 20 times while it ranges from 1 time to 5 times for S4 such that in each pair of datasets, the relative abundance of S. thermophilus LMD-9 in S1 remains four times of that in S4. SA (r=2) was applied to each pair of datasets and the performance of SA was evaluated according to the percentage of extracted reads and the fraction of S. thermophilus genome assembled. As shown in Table 2, although the sequencing depth varies across the simulated datasets, the percentage of extracted reads was significantly correlated with the expected ratio of differential reads (R²=0.9739), indicating that the performance of the subtraction step is mostly determined by the relative abundances of a genome between metagenomes. Furthermore, as shown in FIG. 4, quality of the final assembly is dependent on the sequencing depth. When the sequencing coverage is low (e.g., 4 times), a small proportion of the differential genome can be assembled. However, SA recovers nearly all of the differential positions when the sequencing depth is sufficiently high (e.g., 16 times).

### Example 3

Assembly quality of metagenomes under SA was then tested using another set of simulated metagenomics datasets consisting for five strains of Rhodopseudomonas palustris as shown in Table 3 below.

TABLE 3

| | | |
|---|---|---|
| | | Sequencing |
| Genome | RA¹ | Depth |

| Strain | Length | S1 | S2 | S1 | S2 |
|---|---|---|---|---|---|
| BisA53 | 5,505,494 | 3 | 3 | 18x | 18x |
| BisB18 | 5,513,844 | 3 | 3 | 18x | 18x |
| BisB5 | 4,892,717 | 3 | 3 | 18x | 18x |
| CGA009 | 5,459,213 | 0.1 | 5 | 0.6x | 30x |
| HsA2 | 5,331,656 | 5 | 0.1 | 30x | 0.6x |

¹Relative Abundance. The two samples are S1 and S2.

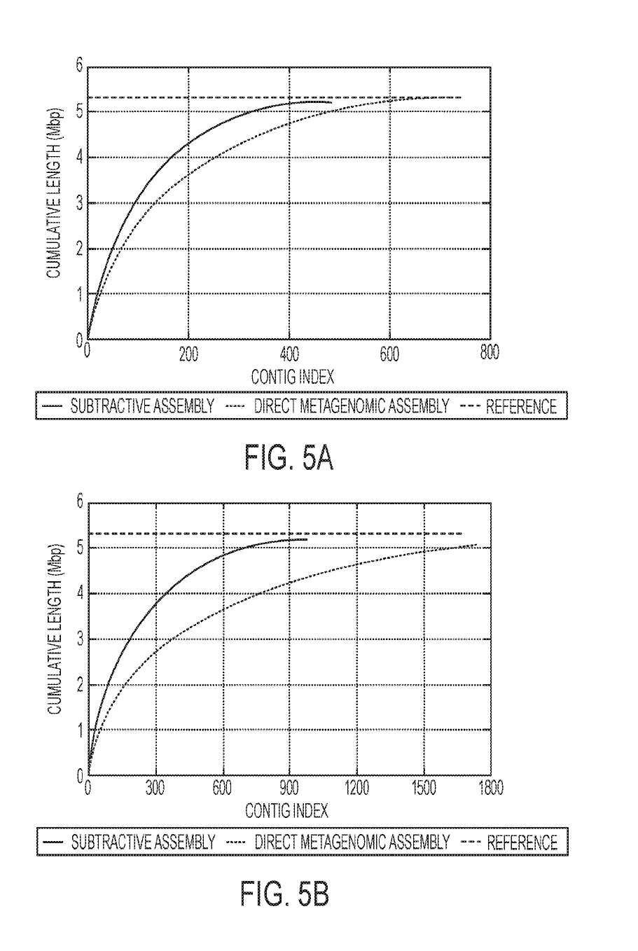

The dominant genome is R. palustris HaA2 in S1, while it is R. palustris CGA009 in S2. At the same time, the relative abundance of R. palustris HaA2 in S2 is substantially lower than that in S1. Thus, k-mers representing the HaA2 genome will be identified and used for extracting reads from S1. For S1, SA obtained longer contigs for the dominant R. palustris HaA2 genome than did direct assembly of the raw datasets, without much sacrifice of genome coverage as shown in FIGS. 5A and 5B. Using contigs that are longer than 500 base pairs, the N50 is 21,374 in SA, compared to 13,360 from the direct metagenomic assembly of metagenome 1; and the length of the largest contig is 113,404 base pairs compared to 95,495 base pairs. The genome coverage by contigs (total number of aligned bases in the reference divided by the genome size) is 98.3% in SA, compared to 98.6% in direct assembly. The increased length of contigs comes with an acceptable number of misassemblies. SA produced three misassemblies (as reported by QUAST), whereas the direct assembly produced one misassembly. The number of mismatches and indels, however, decreased significantly in SA assembly of the distinctive reads: the number of mismatches is 394 with SA and 2,185 with direct assembly; and the number of insertions and deletions (indels) is 8 in SA and 80 in direct assembly.

While not wishing to be bound by this theory, a possible explanation for the superior performance of SA in this simulation is that the subtraction step helps alleviate assembly problems caused by polymorphic regions, where the regions that are similar, but not identical, in multiple genomes in the same metagenomic dataset. The sharing of homologous genes among different species is one of the known complicating factors that confuse de Bruijn graph-based assemblers (including IDBA-UD) in metagenomic assembly, because they form tangled branches in the assembly graph. Since SA targets genomes that are more abundant (or unique) in one of the metagenomes, some of the closely related genomes will be filtered out during the subtraction step, reducing the complexity of the assembly problem.

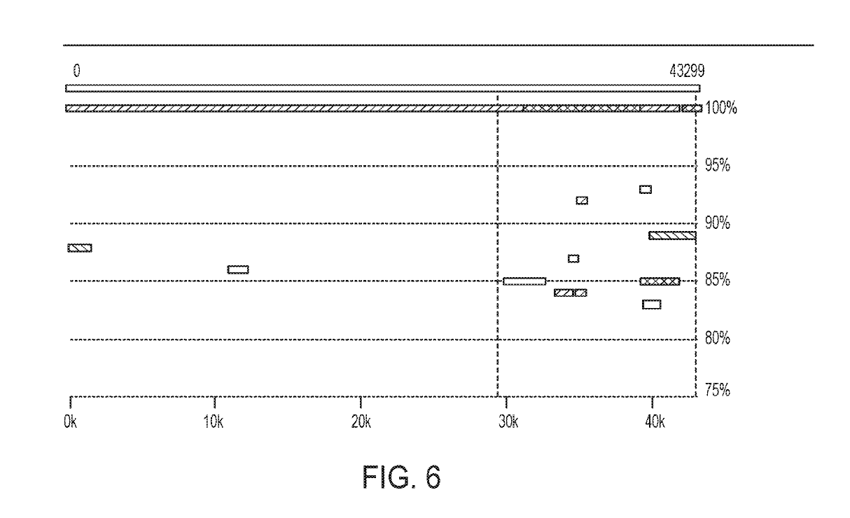

When comparing contigs from SA and direct assembly by NUCMER (included in MUMer package, version 3.0, as described in Delcher, Arthur L. et al. “Fast Algorithms for Large-Scale Genome Alignment and Comparison.” Nucleic Acids Research 30.11 (2002): 2478-2483. Print.), reduced fragmentation of contigs by the SA resulted from the subtraction step. For example, one 43,299-bp contig from subtractive assembly was fragmented into 4 contigs in direct assembly as shown in FIG. 6. Furthermore, a long contig resulting from the subtractive assembly was broken into several shorter contigs when the subtraction step was not used (i.e., from the direct assembly). A number of contigs (annotated with different colors) from the direct assembly are aligned to the long contig with different degrees of similarities, as indicated in FIG. 6. The polymorphic region is highlighted between two vertical dotted lines.

Positions around breakpoints recruited a number of contigs of different degrees of similarities, indicating that these are homologous regions shared by the different genomes in the metagenomic dataset (the five strains are moderately similar to each other at a maximal unique matches index (MUMi) distance of ˜0.8 (0˜1 scale), due to frequent genomic rearrangements).

### Example 4

Assemblies for SA were also tested. In addition to IDBA-UD, MetaVelvet (version 1.1.01, as described in Namiki T., Hachiya T., Tanaka H., Sakakibara Y. (2012). MetaVelvet: an extension of Velvet assembler to de novo metagenome assembly from short sequence reads. Nucleic Acids Res. 40:e155 10.1093/nar/gks678) and MEGAHIT (version 0.2.1, as described in “MEGAHIT: an ultra-fast single-node solution for large and complex metagenomics assembly via succinct de Bruijn graph.” Dinghua Li, Chi-Man Liu, Ruibang Luo, Kunihiko Sadakane, Tak-Wah Lam. Bioinformatics. 2015 May 15; 31(10): 1674-1676. Published online 2015 Jan. 20. doi: 10.1093/bioinformatics/btv033) were also tested. To use MetaVelvet for SA on 51, k-mer length was set to 51 for assembly (as suggested by the MetaVelvet manual). A greater improvement of assemblies was seen by SA since MetaVelvet originally generated shorter contigs for this dataset than did IDBA-UD (FIGS. 5A, 5B), which leaves more room for improvement. From the cumulative plot of contigs, we can see that contigs of the differential genome were longer if SA is applied preceding metagenomic assembly. Using contigs that are longer than 500 base pairs, the N50 is 10,116 in SA, but only 4,681 with direct metagenomic assembly, and the largest contig is increased from 76,007 base pairs in direct assembly to 98,570 base pairs in SA. Even the genome coverage is improved in SA, from 94.1% to 97.6%.

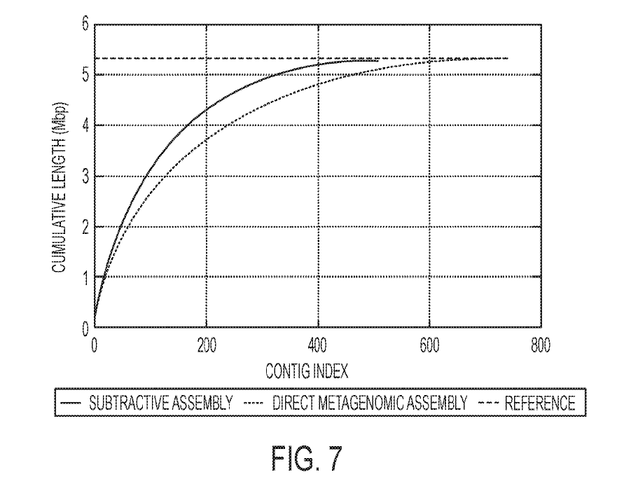

MEGAHIT is a more recently developed assembler, which also uses the iterative assembly strategy similar to IDBA-UD. As shown in FIG. 7, the results of MEGAHIT were comparable to those from IDBA-UD, as shown in FIG. 7, but more differences were observed between these two assemblers for the real T2D gut metagenomes, as discussed below.

### Example 5

SA was applied to the analysis of T2D gut metagenomes in hopes of identifying compositional/functional T2D-associated features of the human microbiome, along with testing SA with datasets of naturally occurring complexity. 50 T2D datasets (a total of 129 gigabases) and all 43 normal glucose tolerant (NGT) datasets (90 gigabases) were used. Three T2D datasets that were outliers were not used, based on neighbor-joining clustering of the samples using a d2 dissimilarity measure for k=9. Table 4 below shows the differential reads extracted for each group of samples (T2D or NGT).

TABLE 4

| k-mer ratio | NGT (Gigabase) | T2D (Gigabase) |
|---|---|---|
| 2 | 14.48 | 12.66 |
| 4 | 2.48 | 1.66 |
| 6 | 0.55 | 0.42 |
| 8 | 0.17 | 0.13 |
| 10 | 0.04 | 0.05 |
| * (unique) | 8.91 | 14.24 |

A large portion of the extracted reads represented unique k-mers, confirming the distinction between these two groups. For comparison, datasets were also assembled directly without the subtractive step of SA. Two different approaches were used for direct assembly: (1) assembling the metagenomic datasets individually, or (2) co-assembling the pooled datasets. For clarity, we call the former direct assembly, and the latter direct co-assembly.

SA generated fewer contigs as compared to the direct coassembly because SA is focused on the differential portion. For direct co-assembly, because the pooled dataset is huge, MEGAHIT was used because of memory space limitations. However, co-assembly of the distinctive reads (i.e., subtractive assembly) for the combined T2D and NGT metagenomes using either assembler was possible, because of the substantial data reduction in the subtraction step. Table 5 shown below summarizes the assembly results for the T2D samples using direct assembly and SA approaches. The results show similar trends for the NGT datasets.

TABLE 5

| | Direct assembly | | | |
|---|---|---|---|---|
| | IDBA-UD | MEGAHIT | Subtractive assembly | |
| Metrics | (individual)² | (co-assembly) | IDBA-UD | MEGAHIT |
| Total contigs ¹ | 2,422,739 | 2,645,944 | 510,220 | 2,175,502 |
| Total base | 3,365,389,115 | 2,280,436,161 | 512,470,294 | 1,434,840,759 |
| N50 | 2,170 | 1,054 | 1,146 | 677 |

¹ Only contigs of at least 300 bps were considered for the statistics.
²The assemblies of individual samples were added for direct assembly by IDBA-UD.

As shown, SAs are approximately one sixth (by IDBA-UD) to one half (by MEGAHIT) the length of direct assemblies, measured as the total length of contigs depending on the assembler used. MEGAHIT produced more contigs, but the contigs produced were much shorter than IDBA-UD contigs, which was the case for both direct assembly and SA. Either assembler may be used in this case. Considering that IDBA-UD gave longer contigs and memory usage is not a concern for SA approach due to the data reduction nature of SA, we have focused below on the downstream application of subtractive assembly results by IDBA-UD was analyzed. However, it is contemplated that users can choose to use an assembler of their preference for SA.

### Example 6

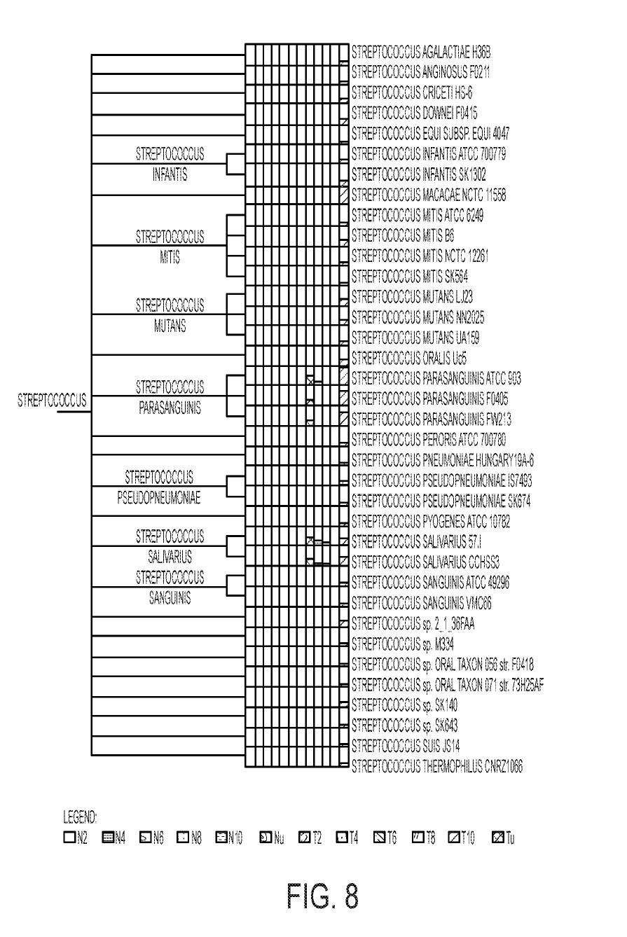

To identify bacteria that compose the difference between T2D and NGT gut metagenomes, contigs from SA were queried against the bacterial genomes (both complete and draft) deposited in NCBI using BLASTN. MEGAN (version 4, as described in Huson, Daniel H. et al. “MEGAN Analysis of Metagenomic Data.” Genome Research 17.3 (2007): 377-386. PMC. Web. 28 Sep. 2016) was used to process the BLASTN search results for taxonomic assignments of the contigs. About one half of the contigs were assigned to a reference genome in the database, and about one third of the unassigned contigs were identified in SA but not in direct assembly. Consistent with previous studies, the results suggested enrichment of Lactobacillus gasseri, Lactobacillus salivarius and Streptococcus mutans in T2D datasets. However, as shown in FIG. 8, a greater variety of Lactobacillus and Streptococcus species was identified as more abundant in the T2D group, as compared to original analyses of these datasets. For example, Streptococcus parasanguinis and Streptococcus salivarius were found to be enriched in the T2D datasets. Genomes that were more abundant in the NGT group were also identified, including Lysinibacillus fusiformis ZC1, Lysinibacillus sphaericus C3-41, and Pseudomonas putida GB-1.

The results also showed that many pathogenic bacteria (including Actinomyces, Enterococcus faecalis and Rothia mucilaginosa) are enriched in T2D datasets, which might be a consequence of the immunocompromised status of T2D patients. The association between enriched pathogens and diabetes has been consistently reported in previous studies: 42 percent of published cases of perianal actinomycosis were from patients also diagnosed with diabetes [36]; diabetes mellitus was identified as a unique, independent risk factor for isolation of vancomycin-resistant E. faecalis [37] and made it easier for R. mucilaginosa to cause infections [38]; and another large-scale metagenomics study revealed higher levels of opportunistic pathogens in participants with T2D.

### Example 7

SA assembly provided genes that could not be well-assembled by direct assembly of individual metagenomic samples, and the previously discussed simulation results show that SA can improve metagenome assembly. SA was further analyzed to examine how it would influence gene prediction and functional analysis results using the T2D datasets. Even though half of the contigs from SA cannot be phylogenetically assigned, the contigs could still be used for functional annotation, which may reduce the bias in the reference-based annotation. When individual samples of subtractive assembly were compared with individual samples of direct assembly (both assembled by IDBA-UD), the following was found. Out of 928,237 genes predicted from SA, 141,104 genes (15%)—among which there were 70,951 complete genes (including both a start codon and a stop codon)—were not found in the direct assemblies of T2D samples. Similarly, 149,321 (18%)—among which 72,956 genes were complete—out of 821,130 genes were not included in the direct assemblies of NGT samples.

Comparison of SA results to the coassembly results of the original datasets (both assembled by MEGAHIT) revealed improvement by SA at comparable scales: 660,445 out of 2,978,267 genes (22%) from SA—among which there are 274,018 complete genes—were not found in the direct co-assemblies of T2D samples. Likewise, 350,997 out of 2,692,810 genes (13%)—among which 132,557 were complete—were not included in the direct co-assemblies of NGT samples. These results suggested that co-assembly of the datasets helped to assemble more genes as compared to assembly of individual datasets, but data reduction by SA helped to further improve the assembly results independent of the assembler used.

When the identified genes were compared to the gene sets from the original analyses of the datasets, a significant number of new genes were found. The original analyses resulted in a collection of 5,997,383 genes from all the samples including NGT samples and T2D samples. Using 95% sequence identity and 80% coverage of the query as the cutoffs, SA resulted in 153,755 new genes (17%) in the T2D group and 140,542 new genes (17%) in the NGT group.

Genes that are unique or more abundant in the T2D microbiomes were annotated according to the SEED classification system as discussed earlier. The gene set shown in Table 6 below is enriched in subsystems including peptidoglycan biosynthesis, multidrug resistance efflux pumps, and lactose and galactose uptake and utilization. These genes are associated with subsystems involved in energy harvesting (such as lactose and galactose uptake and utilization, and fructooligosaccharides and raffinose utilization), cell defense (e.g. peptidoglycan biosynthesis and multidrug resistance efflux pumps), and transport proteins (such as ton and tol transport systems and ECF class transporters), indicating a microbe-contributed elevated level of glycolysis/gluconeogenesis in the T2D group, which is consistent with previous observations that short chain fatty acids can lead to increased glycolysis/gluconeogenesis in the liver. Sialic acid metabolism was also identified as enriched in the gut microbiome of T2D patients as shown in Table 6 below. It has been reported that elevated sialic acid is strongly associated with T2D and raised serum sialic acid is a predictor of cardiovascular complications. As the patients in this study are 70 year old women, they may be in a relatively late stage of diabetes and therefore suffer from those complications.

TABLE 6

| | | # |
|---|---|---|
| Rank | SEED Subsystem | Genes |
| 1 | Peptidoglycan biosynthesis | 635 |
| 2 | Ton and Tol transport systems | 451 |
| 3 | Multidrug resistance efflux pumps | 427 |
| 4 | DNA-replication | 424 |
| 5 | DNA repair, bacterial | 364 |
| 6 | Cell division subsystem | 345 |
| 7 | Lactose and galactose uptake and utilization | 322 |
| 8 | Restriction-modification system | 322 |
| 9 | Fructooligosaccharides and raffinose utilization | 292 |
| 10 | Glycerolipid and glycerophospholipid metabolism | 276 |
| 11 | Maltose and maltodextrin utilization | 270 |
| 12 | Sialic acid metabolism | 257 |
| 13 | Methionine degradation | 253 |
| 14 | Ribosome LSU bacterial | 252 |
| 15 | Glycolysis and gluconeogenesis | 244 |
| 16 | De novo pyrimidine synthesis | 238 |
| 17 | High affinity phosphate transporter | 236 |
| 18 | ECF class transporter | 234 |
| 19 | Purine conversions | 222 |
| 20 | Threonine and homoserine biosynthesis | 220 |

The selection of genes was further narrowed to those that were consistently more abundant across T2D microbiomes, than in healthy controls (so as to serve as dependable gene markers for T2D), considering that SA can improve the assembly of those genes. To identify those consistently-differential genes, the abundance of the genes was quantified using read-mapping by BWA, as described in Li H. and Durbin R. (2009) Fast and accurate short read alignment with Burrows-Wheeler Transform. Bioinformatics, 25:1754-60, normalized by the total number of reads (per billion reads) in each sample, to identify the genes that were significantly enriched in the T2D group compared to NGT group. Among the 141,104 differential genes that were not found in direct assemblies of T2D samples, 18,614 (13%) genes were significantly enriched in all T2D samples, with q-value<0.01 (Wilcoxon rank-sum test corrected by false discovery rate). Although similar rankings of top subsystems were observed, increases of subsystems related to energy harvesting (e.g., the rank for the ‘fructooligosacchrides and raffinose utilization’ subsystem was increased from 7 to 2) were observed using this more stringent collection of T2D differential genes that passed the multiple testing as shown in Table 7 below.

TABLE 7

| | | # |
|---|---|---|
| Rank | SEED Subsystem | Genes |
| 1 | Peptidoglycan biosynthesis | 138 |
| 2 | Fructooligosaccharides and raffinose utilization | 103 |
| 3 | Multidrug resistance efflux pumps | 100 |
| 4 | Cell division | 89 |
| 5 | Sialic acid metabolism | 80 |
| 6 | Gene cluster associated with Met-tRNA | 77 |
| | formyltransferase | |
| 7 | Glycerolipid and glycerophospholipid metabolism | 73 |
| 8 | DNA repair, bacterial | 73 |
| 9 | Choline and bataine uptake and betaine biosynthesis | 73 |
| 10 | Murein hydrolases | 72 |
| 11 | Maltose and maltodextrin utilization | 66 |
| 12 | Beta-glucoside metabolism | 62 |
| 13 | Lactose and galactose uptake and utilization | 62 |

In T2D women, a few enriched SEED subsystems involve utilization of fructooligosaccharides (FOS), maltose, lactose and galactose, which are ranked as 2, 11, and 13 in Table 7. For 11 out of 16 functional roles involved in the ‘Fructooligosaccarides and Raffinose Utilization’ subsystem, genes with differential abundances were identified as shown in Table 8 below.

TABLE 8

| Column | Abbrev | Functional Role | #Hits |
|---|---|---|---|
| 1 | MsmR | MSM (multiple sugar metabolism) operon | 3 |
| | | regulatory protein | |
| 2 | MsmE | Multiple sugar ABC transporter, substrate- | 8 |
| | | binding protein | |
| 3 | MsmF | Multiple sugar ABC transporter, membrane- | 4 |
| | | spanning permease protein MsmF | |
| 4 | MsmG | Multiple sugar ABC transporter, membrane- | 4 |
| | | spanning permease protein MsmG | |
| 5 | MsmK | Multiple sugar ABC transporter, ATP-binding | 6 |
| | | protein | |
| 6 | SacA | Sucrose-6-phosphate hydrolase (EC 3.2.1.B3) | 10 |
| 7 | GtfA | Sucrose phosphorylase (EC 2.4.1.7) | 8 |
| 8 | DexB | Glucan 1,6-alpha-glucosidase (EC 3.2.1.70) | 0 |
| 9 | RafR | Raffinose operon transcriptional regulatory | 0 |
| | | protein RafR | |
| 10 | RafB | Raffinose permease | 0 |
| 11 | LacY | Lactose permease | 0 |
| 12 | Aga | Alpha-galactosidase (EC 3.2.1.22) | 20 |
| 13 | yesM | Two-component sensor kinase YesM | 5 |
| | | (EC 2.7.3.—) | |
| 14 | Man | Alpha-mannosidase (EC 3.2.1.24} | 5 |
| 15 | BG | Beta-glucosidase (EC 3.2.1.21} | 30 |
| 16 | Hyl | Hyaluronoglucosaminidase (EC 3.2.1.35} | 0 |

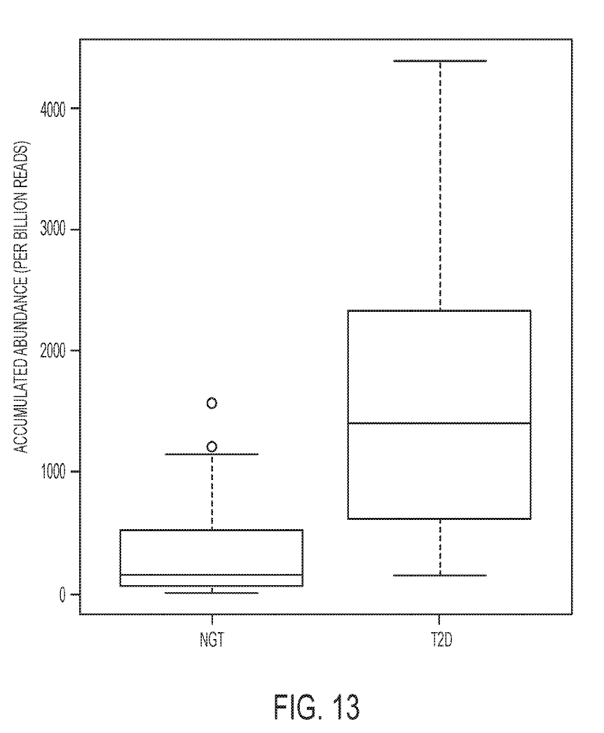

Detailed analysis of FIGfams in these three subsystems revealed an enrichment of several glycosidases with various substrate specificities (EC 3.2.1.-). For the utilization of FOS, there are at least three glycosidases with elevated levels in T2D: beta-glucosidase (EC 3.2.1.21), alpha-galactosidase (EC 3.2.1.22) and alpha-mannosidase (EC 3.2.1.24); for the utilization of lactose and galactose, betagalactosidase (EC 3.2.1.23) is significantly increased in the T2D cohort as shown in FIG. 13.

Similarly, alpha-glucosidase (EC 3.2.1.20) is increased, for an enhanced utilization of maltose. Alpha-glucosidase inhibitors (AGIs) are well-established in the treatment of T2D, and work by reducing the absorption of carbohydrates from the small intestine. SA revealed other enriched glycosidases in T2D, which may provide alternative targets for the development of antidiabetic drugs.

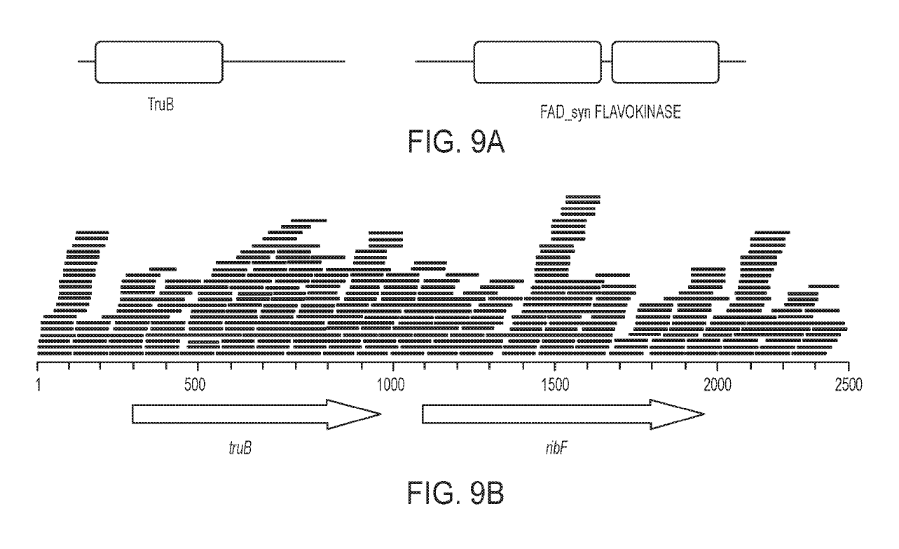

The next two genes, truB (T2D_unique_8729_300_1012_+) and ribF (T2D_unique_8729_1193_2032_+), were found in the same contig assembled by SA. The truB gene encodes the pseudouridylate synthase TruB (PF01509) of 239 amino acids, and the ribF gene encodes a prokaryotic riboflavin biosynthesis protein (PF06574) of 278 amino acids. The gene product of ribF has both flavokinase and adenine dinucleotide synthetase (FAD synthetase) activities (FIG. 9A). FIG. 9A further shows three domains in the operon: TruB encoded by truB; and flavokinase and FAD synthetase encoded by ribF. The flavokinase and FAD synthetase constitute the bifunctional prokaryotic riboflavin biosynthesis protein.

Flavokinases (EC 2.7.1.26) catalyze the conversion of riboflavin to FMN, while FAD synthetase (EC 2.7.7.2) adenylates FMN to FAD, together converting riboflavin to the catalytically active cofactors FMN and FAD. By blasting the genes against the NR database, the source genome was identified as Blautia sp. CAG:257 with 99% identity and 98% coverage of the query sequence. Karlsson et al. also reported an abnormal level of riboflavin metabolism in the gut microbiome of T2D patients; however, they claimed that riboflavin metabolism was enriched in NGT women. The results of Karlsson et al. may actually indicate the opposite (i.e., consistent with the conclusion stated herein): Karlsson et al. identified 3 KEGG protein families (KOs) involved in riboflavin metabolism increased in NGT, while 6 other protein families were more abundant in T2D. See Karlsson F H, Tremaroli V, Nookaew I, Bergstrom G, Behre C J, Fagerberg B, Nielsen J, Backhed F: Gut metagenome in European women with normal, impaired and diabetic glucose control. Nature 2013, 498:99-103; and Kanehisa M, Goto S: KEGG: kyoto encyclopedia of genes and genomes. Nucleic Acids Res 2000, 28:27-30, the disclosures of which are incorporated by reference in their entirety to the extent they are not inconsistent with the explicit teachings of this specification.

The contig containing these genes was assembled from reads ‘unique’ to the T2D samples; read-mapping confirmed that a few reads (59) from the NGT samples can be mapped to this 3,450 base pair contig (in contrast, 521 reads from T2D microbiomes can be mapped to this contig; as shown in FIG. 9B where the genes truB and ribF in this operon are confirmed by reads-mapping; (Reads mapped to the proper pair are colored in blue and mapped singletons are colored in green)).

This increase in FMN and FAD synthetase is consistent with the increased energy harvesting suggested above: FAD helps extract chemical energy by taking electrons from glucose during oxidative respiration.

The last gene (T2D_unique_70674_105_963_+) encodes a 285 amino acids protein with one domain: MATE (PF01554; Multi antimicrobial extrusion protein). The protein belongs to one of the ten protein families (FIGfams) associated with the Multidrug Resistance Efflux Pumps subsystem. This FIGfam (FIG00000402) has the most hits of differential genes (342/427) among the ten FIGfams; members of this protein family extrude cationic drugs through an Na+-coupled antiport mechanism. Taxonomic assignments of these proteins indicate a Firmicutes origin, especially Clostridium, Lachnospiraceae and Erysipelotrichaceae. It is known that mammalian MATE transporters mediate multidrug resistance (MDR) by exporting diverse xenobiotic cations in the liver and kidney (MATE1 protein, for example, reduces the plasma concentrations of metformin, a widely prescribed oral glucose-lowering drug for the treatment of T2D, modulating its therapeutic efficacy), while bacterial MATE transporters act primarily as xenobiotic efflux pumps and have been reported to confer tigecycline resistance. The elevated level of bacterial MATE pumps in the gut of T2D patients suggests a potential link between the disease and the gut microbiome through the elevated levels of medications, including antibiotics, taken by T2D patients.

### Example 8

Two groups of metagenomic datasets were generated using five bacterial genomes from the FAMeS dataset by MetaSim, with each group having a unique population structure as shown in Table 9 below. Ten samples were simulated for each dataset. The two groups of synthetic datasets were compared with a focus on S. thermophilus LMD-9, which has a fold change of 2. Since the S. thermophilus genome is more abundant in population 1 than population 2, we evaluated CoSA (k-mer length of 23) by the proportion of reads extracted and further the assembled genome coverage with the help of QUAST and MUMer.

CoSA detects differential genomes by testing k-mer frequencies with a Wilcoxon rank-sum test rather than by using fold change of k-mers. For comparison purposes, k-mer frequencies are employed concurrently from multiple samples for each group for comparison purposes, which may enable detection of minor but consistent changes between groups of samples. To test the performance of CoSA in such case, simulated metagenomic samples using two population structures as shown in Tables 9-12 below were conducted.

TABLE 9

| | P1^{&} | P2 |
|---|---|---|
| Ferroplasma acidarmanus fer1 | 1^{#} | 1 |
| Lactobacillus gasseri ATCC 33323 | 2 | 2 |
| Pediococcus pentosaceus ATCC 25745 | 4 | 4 |
| Prochlorococcus marinus NATL2A | 8 | 16 |
| Streptococcus thermophilus LMD-9 | 16 | 8 |
| Total Number of Generated Reads | 1,149,971 | 1,147,816 |

^{&}simulated population 1;
^{#}relative abundance of the F. acidarmanus genome in population 1.

As shown above, the Streptococcus thermophilus LMD-9 genome is 2 times more in population 1 (P1) than in population 2 in terms of relative abundance. Similarly, Prochlorococcus marinus NATL2A is the differential genome that is 2 times more abundant in P2 than in P1. Since there was only a fold change of two for the differential genomes, it may be difficult to detect the minor effects through fold change of k-mers. As a result, SA performed relatively poorly on this simulated dataset as discussed herein. As CoSA utilizes k-mer frequencies from each population of multiple samples, we generated 10 samples for each population structure.

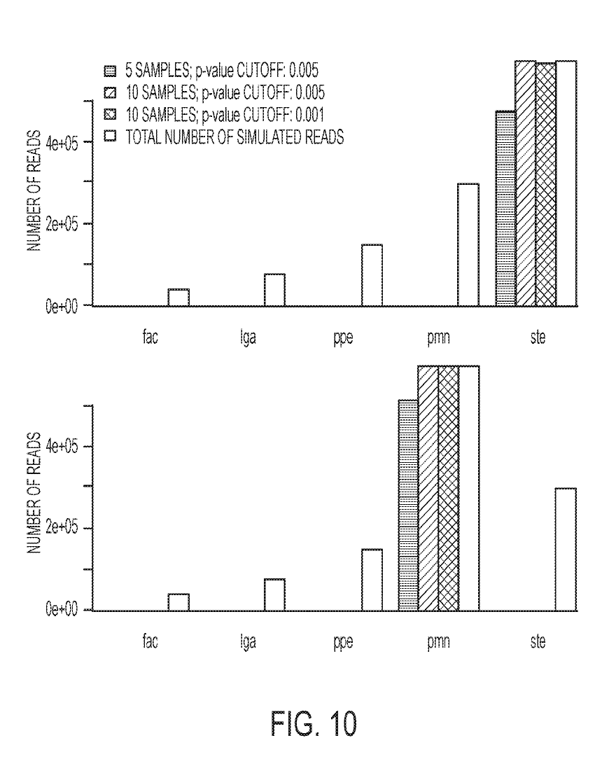

CoSA was evaluated with different parameters, including p-value cut-off and number of samples for each group in comparison as shown in FIG. 10 and Tables 10 and 14 below. One of the parameters includes the efficacy of read extraction using either 5 or 10 samples for each population. As shown in FIG. 10, CoSA extracted more reads from the differential genomes by using more samples. For example, as shown in Table 10 below, 593,739 (99.98%) out of 593,858 short reads were extracted for the S. thermophilus LMD-9 genome from one of the samples by using 10 samples for each population. When 5 samples were used for each population, 471,786 (79.44%) reads were extracted as shown in Table 11 below. Meanwhile, CoSA extracted few reads from the non-differential genomes in both cases.

TABLE 10

| | S1 (P1)^{&} | S1′ (P2) |
|---|---|---|
| Ferroplasma | 0/38,568^{#} | 19/38,569 |
| acidarmanus fer1 | | |
| Lactobacillus gasseri | 122/75528 | 77/76,152 |
| ATCC 33323 | | |
| Pediococcus | 178/146,787 | 25/147,199 |
| pentosaceus | | |
| ATCC 25745 | | |
| Prochlorococcus | 8/295,230 | 587,980/588,579 |
| marinus NATL2A | | |
| Streptococcus | 590,820/593,858 | 0/297,227 |
| thermophilus LMD-9 | | |

^{&}simulated sample 1 using population structure 1;
^{#}0 reads were extracted out of 38,568 reads from the F. acidarmanus genome in S1

TABLE 11

| | S1 (P1)^{&} | S1′ (P2) |
|---|---|---|
| Ferroplasma | 10/38,568^{#} | 116/38,569 |
| acidarmanus fer1 | | |
| Lactobacillus gasseri | 125/75528 | 194/76,152 |
| ATCC 33323 | | |
| Pediococcus | 160/146,787 | 237/147,199 |
| pentosaceus | | |
| ATCC 25745 | | |
| Prochlorococcus | 8/295,230 | 507,808/588,579 |
| marinus NATL2A | | |
| Streptococcus | 471,786/593,858 | 0/297,227 |
| thermophilus LMD-9 | | |

^{&}simulated sample 1 using population structure 1;
^{#}10 reads were extracted out of 38,568 reads from the F. acidarmanus genome in S1

The effects of using a lower p-value cut-off, such as 0.001 as shown in Table 10 above and 0.005 as shown in Table 12 below, were also compared. A lower p-value cut-off reduced the number of extracted reads from both differential and non-differential genomes. Yet, CoSA still extracted most of the reads from the differential genomes. For example, for the S. thermophilus LMD-9 genome, 590,820 (99.49%) out of the 593,858 reads were extracted for the same sample used above. In other words, CoSA effectively extracted reads from differential genomes with a minor fold change whereas minimal number of reads were extracted from non-differential genomes.

Moreover, a stringent pvalue cut-off (e.g., 0.001) works well for this simulated case; however, for real microbiome datasets that have more complex population structure, a less stringent p-value cut-off may be needed for differential reads extraction (because of the sharing of k-mers among species) as shown in the application of CoSA to the T2D microbiomes discussed further herein.

TABLE 12

| | S1 (P1)^{&} | S1′ (P2) |
|---|---|---|
| Ferroplasma | 2/38,568^{#} | 122/38,569 |
| acidarmanus fer1 | | |
| Lactobacillus gasseri | 136/75528 | 546/76,152 |
| ATCC 33323 | | |
| Pediococcus | 193/146,787 | 727/147,199 |
| pentosaceus | | |
| ATCC 25745 | | |
| Prochlorococcus | 8/295,230 | 588,567/588,579 |
| marinus NATL2A | | |
| Streptococcus | 593,739/593,858 | 0/297,227 |
| thermophilus LMD-9 | | |

^{&}simulated sample 1 using population structure 1;
^{#}2 reads were extracted out of 38,568 reads from the F. acidarmanus genome in S1

The assembly quality was also compared for the differential genomes with different numbers of samples by assembling the extracted reads (via QUAST and MUMer). For the S. thermophilus LMD-9 genome in the same sample as described earlier, 95.59% of the reference genome was recovered when 10 samples per population were used, and 73.06% of the genome was assembled when 5 samples were used for each group as shown in FIG. 1. In other words, a higher fraction of genome for the differential genomes was assembled, while also obtaining fewer but longer contigs. Based on contigs of a size greater than or equal to 500 base pairs, we produced 101 contigs with N50 of 34,454 using 10 samples and 1,227 contigs with N50 of 1,219 using 5 samples. With a greater number of samples, CoSA is capable of assembling the differential genomes at a higher quality.

In an alternate assembly quality evaluation with similar parameters, the S. thermophilus LMD-9 genome in the same sample as above, 95.76% of the reference genome was recovered when 10 samples per population were used; but only 73.32% of the genome were assembled when we used 5 samples for each group (as shown in FIG. 14). In other words, a higher fraction of the genome for the differential genomes was assembled, while also obtaining fewer but longer contigs. Furthermore, 84 contigs were produced with N50 of 51,061 using 10 samples and 1,280 contigs with N50 of 1,180 using 5 samples. With a greater number of samples, CoSA is capable of better assembling the differential genomes at a higher quality. By contrast, our original SA approach relies on ratios of k-mers to detect differential reads and only a small fraction (19.64%) of the genome can be assembled using the reads it extracted. That is, CoSA outperforms SA for detecting minor but consistent effect when multiple samples are used, and that using more samples by CoSA results in better assembly of the differential genomes (CoSA-10, 10 samples were used; CoSA-5, 5 samples were used).

Because CoSA employs k-mer frequencies from individual samples, a new dimension for different samples is introduced and therefore increases the requirement of computational resources. To reduce running time and maximum memory usage, CoSA was implemented with multiple threading. Also, counters of k-mers are written on a disk and then loaded back in batches for the detection of differential k-mers. The performance of implementation with varying numbers of both simulated and real metagenomic samples was evaluated as shown in Table 13 below.

TABLE 13

| | Simulation | Real | Real |
|---|---|---|---|
| | (10-10) | (10-10) | (83-98) |
| Total Number of k- | 9,112,554 | 1,660,238,449 | 6,225,603,794 |
| mers | | | |
| Running Time | 9 m 25 s | 2 h 27 m 10 s | 49 h 23 m |
| Maximum Memory | 2 G | 51 G | 192 G |

CoSA extracted reads within 10 minutes for small number (˜10) of samples with several millions of k-mers while it took a couple of days for comparing moderate number (˜100) of real metagenomic samples with billions of k-mers. In terms of memory usage, CoSA requires 192 gigabytes memory when we were comparing 98 gut metagenomes from patients with 83 samples from healthy controls, which is acceptable in modern computing clusters.

### Example 9

CoSA was applied to two cohorts of gut microbiome associated with type II diabetes, and liver cirrhosis, respectively. The T2D cohort was derived from two groups of 70-year-old European women, one group of 53 with T2D and the other was derived from a matched group of healthy controls (NGT group; 43 participants). The SA approach was tested using the T2D cohort, and in this study, the comparison of CoSA with SA using the T2D datasets was analyzed. The liver cirrhosis cohort contains datasets from 123 patients with liver cirrhosis and 114 healthy individuals of Han Chinese origin. This cohort is used to showcase the application of CoSA. The metagenomes were divided into discovery (or training) data and validation data. In discovery phase, 98 patients with liver cirrhosis were compared with 83 healthy controls, while the additional 25 patients and 31 controls were utilized in the validation phase.

The T2D cohort was mainly used to demonstrate the performance of CoSA and compare it with SA. The liver cirrhosis cohort was mainly used to demonstrate the application of CoSA for identification of disease-associated sub-microbiome.

As discussed earlier, CoSA was able to detect minor, but conserved differential genomes using the simulated datasets. In this Example, the T2D microbiome cohort was used to demonstrate the advantages of CoSA. As shown in Table 14 below, CoSA has resulted in a greater reduction of the sequencing data than the original SA reads. CoSA retained 4.88% of the total bases while SA retained 22.66% of the original sequencing data, and CoSA assembled differential genes more efficiently than the SA method.

TABLE 14

| | SA | CoSA |
|---|---|---|
| Total base pairs of | 29.16 GB | 6.30 GB |
| extracted reads | | |
| Reduction rate (total | 22.6% | 4.9% |
| extracted base | | |
| pairs/total base pairs) | | |
| Total genes | 928,237 | 21,084 |
| Differential genes | 92,239 | 14,770 |
| Enrichment ratio | 9.9% | 71.0% |

### Example 10

Because CoSA is sensitive in the detection of differential compositions between two groups of samples, it can be used to identify biomarkers for certain factors (e.g., diseased vs. healthy) by comparing metagenomes. In this example, CoSA is applied to detect differential genomes/genes in gut microbiome of liver cirrhosis cohorts. The samples of the datasets were divided into two phases: 181 samples (98 patients and 83 healthy controls) for discovery phase and 56 samples (25 patients and 31 healthy controls) for validation phase. Based on the differential genes, a classifier that could accurately identify patients with liver cirrhosis was trained.

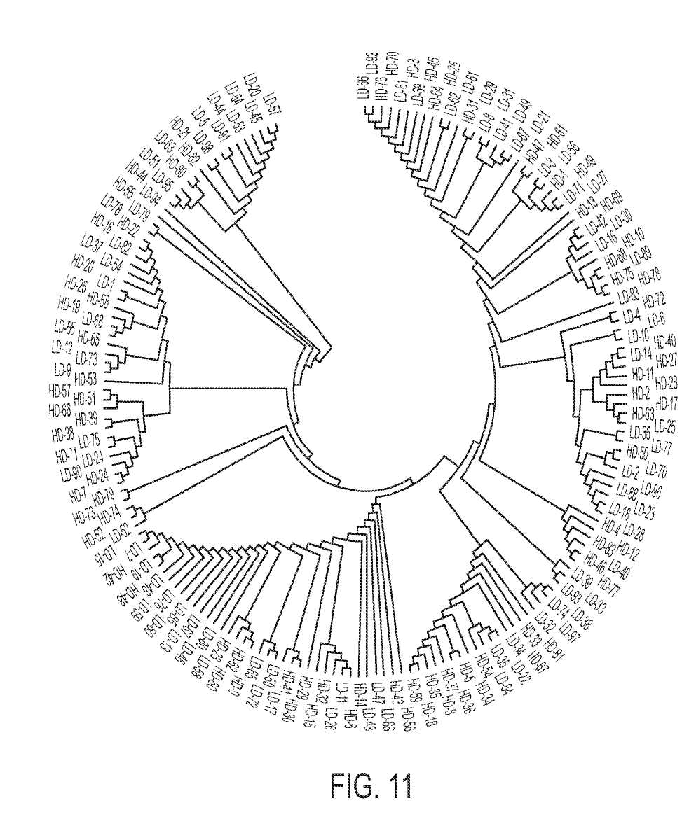

When comparing metagenomes, extensive variations across samples should be taken into account, including temporal dynamics within individuals and variations from different patients. Currently, most available data sets do not account for those variations and therefore, it is difficult to identify differential genes or biomarkers. Before identifying biomarkers from the gut microbiome in liver cirrhosis, the relationship of the metagenomes by a Neighbor-Joining clustering analysis of the discovery data sets was investigated as shown in FIG. 11. The distance matrix for the clustering was calculated based on 9-mers by d2Meta. HD refers to healthy controls and LD stands for liver cirrhosis patients used for discovery phase. Although there were small clusters of diseased or healthy samples, classification between the gut microbiome from patients with liver cirrhosis and healthy individuals was unclear. While liver cirrhosis may not have the power to alter the microbial compositions, there should be microbes enriched in patients with liver cirrhosis to form those clusters for patients.

Compositional analysis of the contigs assembled revealed species that are enriched in liver cirrhosis microbiome, consistent with previous results. In addition, Klebsiella pneumoniae was found to be present in patients' gut microbiomes. Based on the data, specific genes were predicted from the assembled contigs, the abundances of the genes were quantified, and the genes that were differentially abundant in the normal versus diseased microbiomes were annotated. In total, 95,718 differential genes were predicted, which were further used for building a predictor for disease using microbiome data.

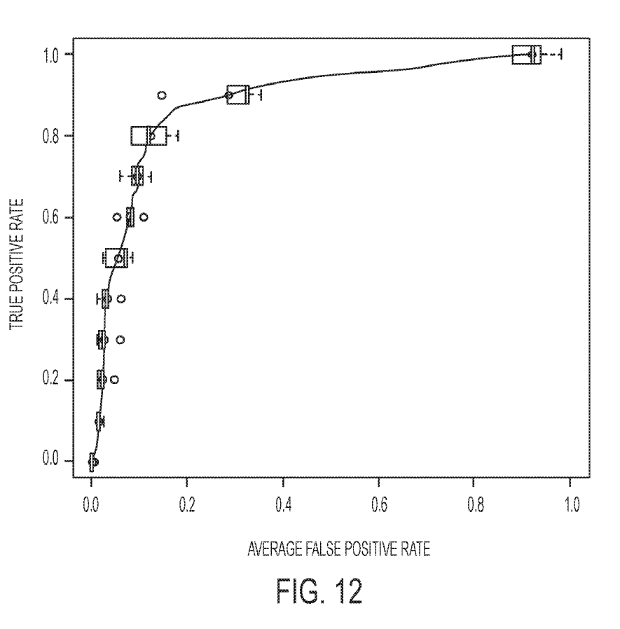

With the 95,718 differential genes, support vector machine (SVM) discriminators were trained using the discovery samples. The SVM algorithm used was coupled with recursive feature elimination (RFE), which could automatically determine the optimal number of features by the span estimate. This was repeated 5 times of 10-fold cross validation while training SVM classifiers as shown in FIG. 12, achieving an area under the receiver operating characteristic curve (AUC) value of 0.889 on average.

### Example 11

In this Example, CoSA was applied to the T2D microbiome cohort. As shown in Table 15 below, CoSA has resulted in a greater reduction of the sequencing data (CoSA retained 8.99% of the total bases) than the original SA reads (SA retained 17.59% of the original sequencing data). Extracted reads were then used for assembly and gene annotation. Although reads extraction by CoSA resulted in a smaller collection of microbial genes than the SA approach (since CoSA retained much fewer reads than SA), genes from CoSA tend to be more consistently differential across the samples between the groups. Genes derived from CoSA (1,008,068 genes) and SA (1,648,016 genes) were pooled, resulting a collection of 2,656,084 genes, and the abundances of the genes in this collection were quantified. The gene abundance profile was then used for WRS test between the patient and the healthy control groups, with correcting for multiple testing using false discovery rate (q-value) computed by the tail area-based method of the R fdrtool package [41]. Table 15 summaries the test results, indicating that CoSA produced a greater number of significantly differential genes than SA. None of the genes derived by SA had q-value less than 0.05.

TABLE 15

| | CoSA [symbol] | SA |
|---|---|---|
| Total base pair in extracted reads | 11.59 Gbp | 22.68 Gbp |
| | (8.99%) | (17.59%) |
| # of predicted genes [symbol] | 1,008,068 | 1,646,016 |
| # of significant genes (q-value ≤ 0.07) | 563,743 | 285, [symbol] 66 |
| # of significant genes (q-value ≤ 0.05) | 357,591 | 0 |

[symbol]

p-value = 0.2 and voting threshold = 0.8 were used for reads extraction [symbol]
only counted genes assembled from extracted reads from patients (but not healthy individuals). [symbol]
indicates data missing or illegible when filed

### Example 12

CoSA was applied to T2D datasets (including datasets from patients and healthy individuals) using different settings of parameters and compared the performance of classifiers built from the assembled microbial genes (from both T2D patients and healthy-controls). Table 16 below summarizes the results. Two different classification algorithms were used. One was SVM (Support Vector Machine) with linear kernel and the other is RF (Random Forest) whose forest included 10 trees. Using p-value of 0.05 and voting threshold of 0.3 (referred to as “Normal” in Table 16) for reads extraction in CoSA followed by assembly and abundance quantification, 296,979 genes were derived. The collection of genes resulted in a SVM that achieved a prediction accuracy of 0.94 (AUC).

CoSA was also tested using more stringent parameters for reads extraction (p-value=0.001 and voting threshold=0.5). The reads extraction resulted in a small reads file with 19.13Mbp in total. 249 genes were assembled and predicted from this collection of sequencing reads, and an RF model (without using feature selection) built from this small set of microbial genes achieved an AUC of 0.79. The advantage of using this setting (referred to as “Strict” in Table 16) is that only a small number of reads were extracted and only a small number of genes need to be quantified and used for building classifiers, and a reasonable prediction accuracy was still achieved.

Further, CoSA was applied using a looser setting (p-value=0.2 and voting threshold=0.8; referred to as “Loose” in Table 16), which resulted in the extraction of many more reads, and many more genes can be assembled. However, a greater number of genes at the beginning does not necessarily result in a better classifier for prediction. The best classifier built using the larger collection of genes achieved an AUC of 0.89. Similarly, using our original subtractive assembly approach (SA), an even greater collection of microbial genes can be assembled. However, the best predictor built using this larger collection of genes only achieved an AUC of 0.85 as shown in Table 16.

TABLE 16

| | | CoSA | | | |
|---|---|---|---|---|---|
| | | Strict | Normal | Loose | SA |
| Reads extraction | P-value cut-off | 0.001 | 0.05 | 0.2 | —^{a} |
| | Voting threshold | 0.5 | 0.3 | 0.8 | 0.5 |
| | Total base pair | 19.13 Mbp | 6.08 Gbp | 19.23 Gbp | 36.26 Gbp |
| Classification | # of genes | 249 | 296,979 | 1,741,472 | 2,098,590 |
| | # of genes selected^{b} | 249^{c} | 207 | 230 | 210 |
| | Classifier | RF | SVM | SVM | SVM |
| | AUC^{d} | 0.79 | 0.94 | 0.89 | 0.85 |

^{a}SA uses ratios of k-mer counts to determine differential k-mers;
^{b}genes were selected using L1-based feature selection method;
^{c}no feature selection was applied for this case;
^{d}average accuracy using 10-fold cross-validation.

In accordance with the present disclosure, CoSA efficiently extracts reads that are likely sequenced from differential genes across samples for the identification of conserved microbial marker genes.

The time and space complexity of CoSA is related to the number of datasets and the size of each dataset. The running time and memory cost is small for small datasets such as the simulated microbiome datasets. However, the computational time and memory usage can be substantial for large cohorts of datasets such as the T2D datasets. For example, the total running time of CoSA for the simulated datasets was 44 minutes (38 minutes for k-mer counting and 6 minutes for the detection of differential k-mers and therefore differential reads), and the peak memory usage was 2G.

However, for the large T2D cohort, the running time for k-mer counting was 6.9 hours and the next step of detecting differential k-mers and reads took 27.5 hours. The peak memory usage for the T2D datasets was also substantial, which was 229 Gb. In the current implementation of CoSA, WRS test is applied to k-mer counts normalized by the total k-mers (which is equivalent to the total reads) in each sample, for the detection of k-mers with differential abundances across healthy controls and patients.

While this disclosure has been described as having an exemplary design, the present disclosure may be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains.

Furthermore, the connecting lines shown in the various figures contained herein are intended to represent exemplary functional relationships and/or physical couplings between the various elements. There are many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U.S.C. § 112(f), unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

What is claimed is:

1. A method for characterizing disease-associated microbial marker genes comprising:
counting k-mers from groups of samples;
loading k-mers into a hash table;
loading k-mers into a two-dimensional array to detect differential k-mers;
extracting reads based on differential k-mers; and
assembling contigs from the reads with an assembler and annotating the contigs.

2. The method of claim 1, wherein a Wilcoxon Rank Sum test is used to identify differential k-mers between the groups of samples.

3. The method of claim 1, wherein the extracting reads step includes utilizing a voting threshold to characterize whether the read is a differential read.

4. The method of claim 3, further comprising assembling extracted reads into contigs.

5. The method of claim 3, wherein the assembler is IDBA-UD, MetaVelvet or MEGAHIT.

6. The method of claim 4, further comprising predicting protein coding genes from the contigs using a program.

7. The method of claim 5, further comprising estimating the abundance of the predicted coding genes.

8. The method of claim 5, further including pooling contigs for differential samples and removing the redundancies.

9. The method of claim 6, further comprising creating a classifier to discriminate diseased patients from healthy patients.

10. A method for characterizing disease-associated microbial marker genes comprising:
selecting at least two different microbiomes;
counting k-mers from the at least two different microbiomes;
using a k-mer count ratio to generate differential k-mers;
employing differential k-mers to extract distinctive reads;
extract distinctive reads with an assembler;
assembling and phylogenetically annotating contigs from the extracted reads; and
predicting coding genes from the contigs.

11. The method of claim 10, wherein the k-mer count ratio is between 2 and 10.

12. The method of claim 11, wherein the employ differential k-mer step is iterative including a minimum k-mer ratio of 2, a maximum k-mer ratio of 10, and a step value of 2.

13. The method of claim 10, wherein the assembler is IDBA-UD, MetaVelvet or MEGAHIT.
